(19) United States
(12) Patent Application Publication
(10) Pub. No.: US 2009/0232710 A1
(43) Pub. Date: Sep. 17, 2009

(54) Ionic air purifier

(76) Inventor: William Kinsey, Valencia, CA (US)

Correspondence Address: CHARLES R. SUTTON,;LAW OFFICES OF ROGER C. HSU, 201 S. LAKE AVE., STE.,600, PASADENA, CA, 91101, US

(21) Appl. No.: 12/077,236

(22) Filed: Mar. 17, 2008

(51) Int. Cl. A61L 9/03

(52) U.S. Cl. 422/119; 422/125

(57) ABSTRACT

An air freshener to plug into a standard automobile cigarette lighter socket or alternatively the air purifier may be equipped with batteries so it can be left inside a refrigerator thereby freshening the air inside the refrigerator. A third alternative would be to have a power cord adapted to use an external power source, such as through the USB port of a computer or the alternating current wall socket of a home or office. The negative ion generator is in the form of a pin. The negative ion generator produces the negative ions to purify the air. The device can have a light sensor to activate the light when light is low. The device can also come with light emitting diodes which can be set to indicate the status of the various systems of the device. The device can come equipped with an air freshener that disperses vaporized perfume into the air. It also may come equipped with a switch to turn the ion generator or the light, or both on or off. It may be able to fold at one or more points in order to make its presence in the automobile cab as unobtrusive as possible (for example so it doesn't hinder the use of a gear shift knob). The device comes equipped with a switch and a joint at which point it can be folded.

### CROSS REFERENCE TO RELATED APPLICATIONS

There are no related applications.

### STATEMENT REGARDING FEDERALLY SPONSORED RESEARCH OR DEVELOPMENT

This invention was not made using federally sponsored research and development. The inventor retains all rights.

### BACKGROUND OF THE INVENTION

There are numerous inventions generating negative ions for one purpose or another, many of which claim both a positive and a negative pole or have a brush shaped ion generator. Excessively complex generators can greatly reduce life span of the device because, frankly, difficulties in cleaning generally result in no cleaning. Cleaning techniques involving the trimming of brush filaments will obviously result in reduction of the brush filament surface area available. Eventually the ion generating surface is either so oxidized or corroded that the number of ions emitted is substantially reduced or else the ion generating surface is reduced to nothing by the act of trimming. Either way a new device becomes necessary. Frequently generating negative ions is for the purpose of cleaning the air. Other devices exist which provide fragrances for the air, thus masking disagreeable odors. Sometimes these fragrance distributors are combined with a plug in night light. These devices do not actually clean the air of underlying odor causing agents. They simply add other chemicals into the air. Other devices exist which feature a night light that can be automatically activated in the event there is no illumination. The light is switched on automatically at night. Negative ions have been proven to remove the following particles from the air, resulting in healthier living conditions and reduction of odors: animal dander, toxic chemicals, bacteria, dust mites, dead skin, smoke, pollen, dust, insect feces, and mold spores. Negative ions in the air can vitalize the human body as though one is in a forest, near a waterfall, or near a hot spring. Inhaled, negative ions accelerate the metabolism, promote vitality, enhance blood flow, stabilize the nervous system, and help fight fatigue. They also increase alertness, reduce asthma and allergy symptoms, and improve sleep.

### BRIEF SUMMARY OF THE INVENTION

A negative ion generator having a power source such as a plug adapted to be inserted in the cigarette lighter socket of a vehicle so the cab interior air will be freshened. Alternatively the air purifier may be equipped with batteries so it can be left inside a refrigerator thereby freshening the air inside the refrigerator. A third alternative would be to have a power cord adapted to use an external power source, such as through the USB port of a computer or the alternating current wall socket of a home or office. The negative ion generator can have a switch to turn it on or off and the preferred embodiment can generate about 1.2 million negative ions per cubic centimeter. The ions attach themselves to numerous unwanted particles such as bacteria and dust, and remove them from the air.

### BRIEF DESCRIPTION OF THE SEVERAL VIEWS OF THE DRAWINGS

FIG. 1 is a perspective view of the auto freshener embodiment in extended position

FIG. 2 is another perspective view of the auto freshener embodiment in an extended position

FIG. 3 is a perspective view of the auto freshener embodiment folded at its point of articulation.

FIG. 4 is a perspective view of the refrigerator freshener embodiment

FIG. 5 is a perspective view of the office freshener embodiment

### DETAILED DESCRIPTION OF THE INVENTION

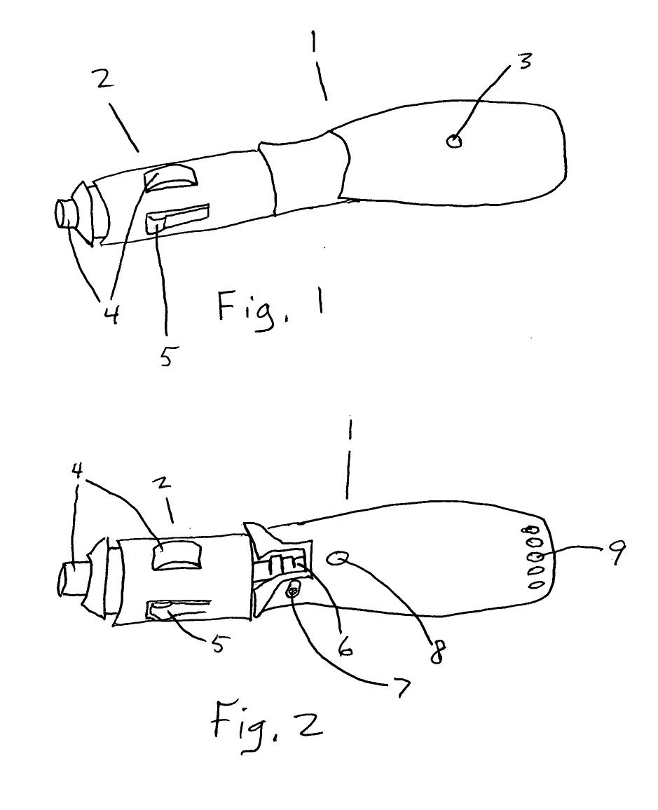

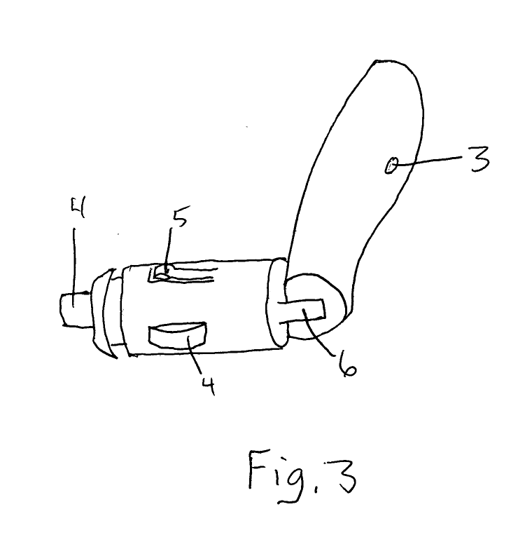

A negative ion generator having power source which can be a plug adapted to be inserted in the cigarette lighter socket of a vehicle so the cab interior air will be freshened. Alternatively the air purifier may be equipped with batteries so it can be left inside a refrigerator thereby freshening the air inside the refrigerator. A third alternative would be to have a power cord adapted to use an external power source, such as through the USB port of a computer or the alternating current wall socket of a home or office. The negative ion generator can have a switch to turn it on or off and the preferred embodiment can generate about 1.2 million negative ions per cubic centimeter. A negative ion generator in the form of a pin is in the preferred embodiment. The pin design can be easily cleaned with a swab and cleaning solution such as rubbing alcohol, eliminating the need to trim or clean a brush electrode when corrosion or deposits form. This represents an improvement over prior art devices which present cleaning difficulties that result in limited useful life span of the device. In one embodiment the negative ion generator is always on when the device is in a socket or plugged into a power source and produces the negative ions to purify the air. The device may have a light and can have a light sensor to activate the light when light is low. The device can also come with light emitting diodes which can be set to indicate the status of the various systems of the device. In the preferred embodiment of the car freshener embodiment, these diodes are, in fact, the light which the invention comes with. It also may come equipped with a switch to turn the ion generator or the light, or both on or off. It may be able to fold at one or more points in order to make its presence in the automobile cab as unobtrusive as possible (for example so it doesn't hinder the use of a gear shift knob). The device can come equipped with an air freshener that disperses vaporized perfume into the air. It has a control circuit connected to the power source, a negative ion generator in the form of an easily cleanable pin, and a voltage amplification circuit that outputs to the negative ion generating pin. The ions that are generated are negative. They result in a breaking down of the ions in the air either to positive or negative. The negative ions that are generated by the device are convected and cause a binding of the heavier positive ions (which are undesirable) so that they fall to the floor or are deposited on the pin. These positive ions are undesirable because they are unhealthful and/or cause odor. Referring now to FIG. 1, in the preferred embodiment of the car freshener we see a car air freshener (1) having a plug (2) adapted to fit into an automobile cigarette lighter socket. There is a light (3) which may be a light emitting diode. Circuit engaging means (4) are present through which the invention can draw power from the electrical system of the automobile. Bracing means (5) may also be available which are adapted to hold the device securely in the cigarette lighter socket. Referring now to FIG. 2, we see that a movable articulated joint (6) can be available that works like a hinge and allows the device to be folded. This joint (6) can be attached to the device by attachment means (7) such as bolts. A switch (8) may be present by which the systems of the invention can be controlled. Vents (9) allow the negative ions generated by the device to escape into the atmosphere of the automobile cabin.

### Functions and Features of the Refrigerator Embodiment

The refrigerator air purifier embodiment can fill the refrigerator with negative ions and kill bacteria including Escherichia coli, Pseudomonas fluorescens, and Bacillus subtilis on food, vegetables and fruits.

Negative ions can eliminate the strong smells, eliminate the pesticides which remain on vegetables and fruits even after washing and keep foods fresh.

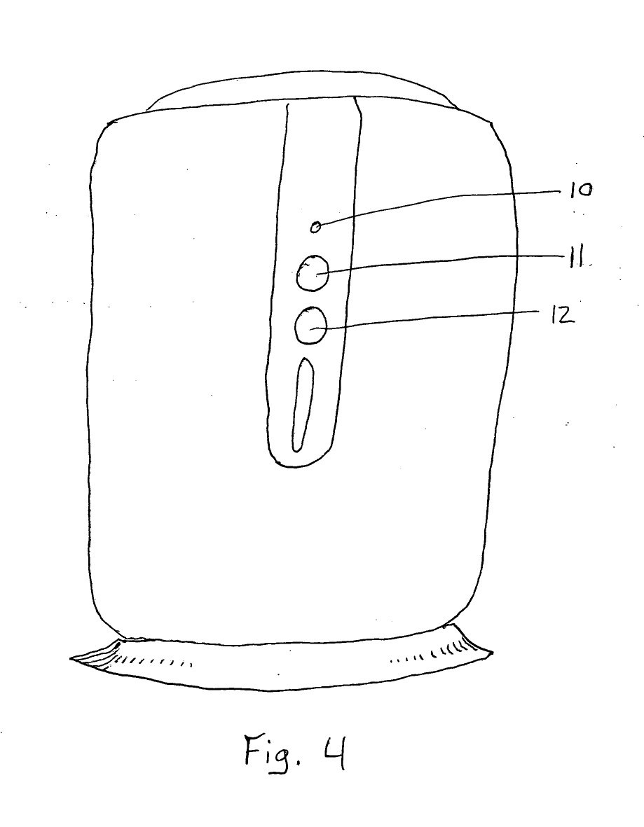

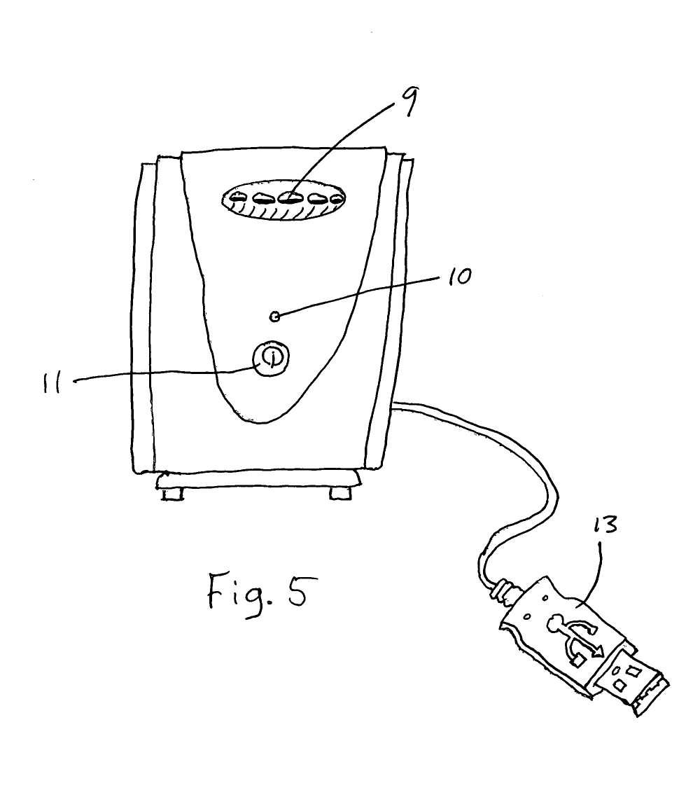

Referring now to FIG. 4, A built-in micro CPU intelligently controls the operation cycles. When the negative ion generator is operating, the indicator will blink every 3 seconds (as an example), when not working, the power indicator will link every 10 seconds (as an example).

How to Operate

Remove the battery door on the bottom of the unit, install 4 A 1.5V high-quality alkaline batteries (although other forms of batteries can be used, these are the type used in the preferred embodiment).

Press the ON/OFF button once and the power indicator lights up. You will see the blink every 3 seconds which means the unit is working.

Press the MODE button to choose the different power settings.

MODE ONE: Press the MODE button, the power indicator will light up GREEN (as an example). The unit first works continuously for about 30 minutes (as an example). The unit will then go into an automatic cycle of a 4-minute operation (as an example) and deodorize every 2 hours (as an example).

MODE TWO: Press the MODE button the second time and the power indicator will light up orange (as an example). The unit first works continuously for about 20 minutes (as an example). Then the purifier will go into an automatic cycle with a 2 minutes operation every hour (as an example).

For best results

1) Select MODE TWO (orange) when the refrigerator capacity is greater than 250 liters or has a strong smelly odor in the refrigerator.

2) Place the air purifier in the refrigerator top compartment for better results.

Turning now to FIG. 4, the version of the invention that can be used in an office, the invention comes equipped with vents (9), an indicator (10, an on/off button (11), and a uniform serial bus (USB) plug (13).

I claim:

1. An ion generating assembly comprising:
a plug adapted to fit into a standard automobile cigarette lighter; said plug being operatively connected to a control circuit, a voltage amplification circuit, and an ion generator; said ion generator having a pin to produce ions.

2. The ion generating assembly of claim 1 having a switch.

3. The ion generating assembly of claim 1 having a light sensor and a light; said light sensor being connected to said control circuit and adapted to activate said light in the event of low ambient light conditions.

4. The ion generating assembly of claim 3 further comprising means to vaporize chemicals having a pleasant scent and distribute said chemicals outside said assembly.

5. The ion generating assembly of claim 3 further comprising light emitting diodes visible from outside said assembly; said light emitting diodes being connected to said control circuit; and said light emitting diodes providing information about the status of said control circuit.

6. The ion generating assembly of claim 5 further comprising means to vaporize chemicals having a pleasant scent and distribute said chemicals outside said assembly.

7. An ion generating assembly comprising:
a decorative housing having ports; a plug; said plug being operatively connected to a control circuit, a voltage amplification circuit, and an ion generator; said ion generator having a pin to produce ions; said ions exiting said decorative housing through said ports.

8. The ion generating assembly of claim 7 in which said plug is a universal serial bus.

9. An ion generating assembly comprising:
an internal power source; said internal power source being operatively connected to a control circuit, a voltage amplification circuit, and an ion generator; said ion generator having a pin to produce ions.

10. The ion generating assembly of claim 9 having a housing, said housing having ports through which said ions can exit said ion generating assembly.

11. The ion generating assembly of claim 10 having a switch.

12. The ion generating assembly of claim 11 having a light sensor and a light; said light sensor being connected to said control circuit and adapted to activate said light in the event of low ambient light conditions.

13. The ion generating assembly of claim 12 further comprising light emitting diodes visible from outside said assembly; said light emitting diodes being connected to said control circuit; and said light emitting diodes providing information about the status of said control circuit.

15. The ion generating assembly of claim 13 further comprising means to vaporize chemicals and distribute said chemicals outside said assembly.
